(19) United States
(12) Patent Application Publication
(10) Pub. No.: US 2023/0131947 A1
(43) Pub. Date: Apr. 27, 2023

(54) Sensor and Method of Manufacturing Sensor

(71) Applicants: Measurement Specialties (China) Ltd., Shenzhen City (CN); Measurement Specialties (Chengdu) Ltd., Chengdu (CN)

(72) Inventors: Baoliang Li, Shenzhen (CN); Benxiang Wang, Shenzhen (CN); Xiao (Kevin) Wu, Chengdu (CN)

(73) Assignees: Measurement Specialties (China) Ltd., Shenzhen City (CN); Measurement Specialties (Chengdu) Ltd., Chengdu (CN)

(21) Appl. No.: 17/847,289

(22) Filed: Jun. 23, 2022

(30) Foreign Application Priority Data
Jun. 25, 2021 (CN) 202110710279.2

(51) Int. Cl. A61B 5/1455; A61B 5/00

(52) U.S. Cl. CPC A61B 5/14552; A61B 5/6832; A61B 2562/12

(57) ABSTRACT

A sensor includes a sealing tape having a first folding line and a second folding line, and an electronic component adhered to a middle area between the first folding line and the second folding line. A first side area of the sealing tape is folded along the first folding line and adhered to the middle area to cover the electronic component. A second side area of the sealing tape is folded along the second folding line and adhered to the first side area to hermetically wrap the electronic component in the sealing tape.

### CROSS-REFERENCE TO RELATED APPLICATION

This application claims the benefit of the filing date under 35 U.S.C. § 119(a)-(d) of Chinese Patent Application No. 202110710279.2, filed on Jun. 25, 2021.

### FIELD OF THE INVENTION

The present invention relates to a sensor and a method of manufacturing the sensor.

### BACKGROUND

In the prior art, a blood oxygen sensor usually includes a light emitter, a light receiver, and a circuit board. The light emitter and the light receiver are electrically connected to the circuit board. The light emitter is used to emit light, and the emitted light is received by the light receiver after passing through the blood. The oxygen saturation of the blood can be analyzed according to the received light.

In the prior art, in order to seal and isolate the light emitter, the light receiver and the circuit board from the blood, a waterproof paper and an adhesive are usually pasted on the light emitter, light receiver and circuit board layer by layer manually. Due to the high height of the light emitter and the light receiver, an opening will appear at the surrounding edge of the pasted waterproof paper, which will lead to poor sealing and affect the waterproof nature of the product. In addition, the manual operation cost is high and is inefficient in producing large quantities.

### SUMMARY

A sensor includes a sealing tape having a first folding line and a second folding line, and an electronic component adhered to a middle area between the first folding line and the second folding line. A first side area of the sealing tape is folded along the first folding line and adhered to the middle area to cover the electronic component. A second side area of the sealing tape is folded along the second folding line and adhered to the first side area to hermetically wrap the electronic component in the sealing tape.

### BRIEF DESCRIPTION OF THE DRAWINGS

The invention will now be described by way of example with reference to the accompanying Figures, of which:

FIG. 1 is a schematic perspective diagram of a sealing tape according to an embodiment;

FIG. 2 is a schematic perspective view of a plurality of electronic components on a middle area of the sealing tape;

FIG. 3 is a schematic perspective view of folding and adhering a first side area of the sealing tape to the middle area to which the electronic component has been adhered;

FIG. 4 is a schematic perspective view of folding and adhering a second side area of the sealing tape to the first side area;

FIG. 5 is a schematic perspective view of two pieces of sub adhesive tapes of a sealing tape according to another exemplary embodiment of the present invention; and

FIG. 6 is a schematic perspective view of a sealing tape bonded by two pieces of sub adhesive tapes shown in FIG. 5.

### DETAILED DESCRIPTION OF THE EMBODIMENTS

Exemplary embodiments of the present disclosure will be described hereinafter in detail with reference to the attached drawings, wherein like reference numerals refer to like elements. The present disclosure may, however, be embodied in many different forms and should not be construed as being limited to the embodiments set forth herein; rather, these embodiments are provided so that the present disclosure will convey the concept of the disclosure to those skilled in the art.

In the following detailed description, for purposes of explanation, numerous specific details are set forth to provide a thorough understanding of the disclosed embodiments. It will be apparent, however, that one or more embodiments may be practiced without these specific details. In other instances, well-known structures and devices are schematically shown to simplify the drawing.

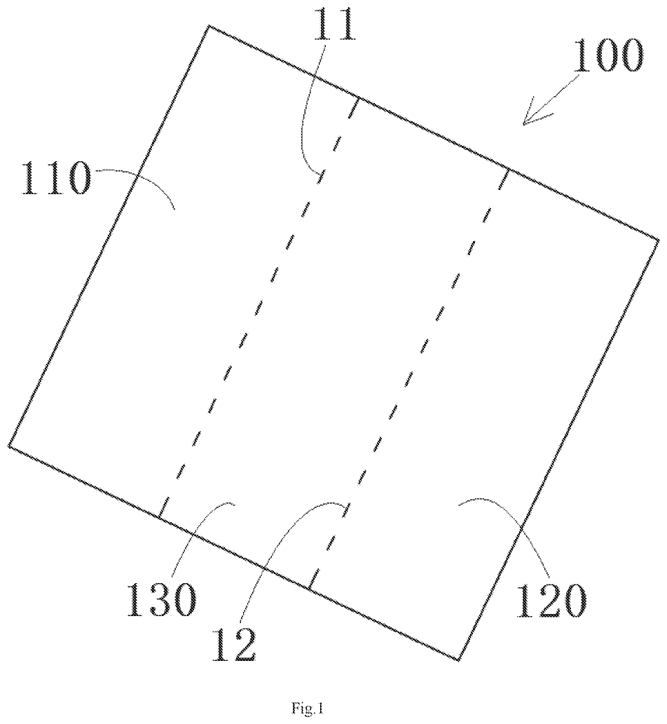

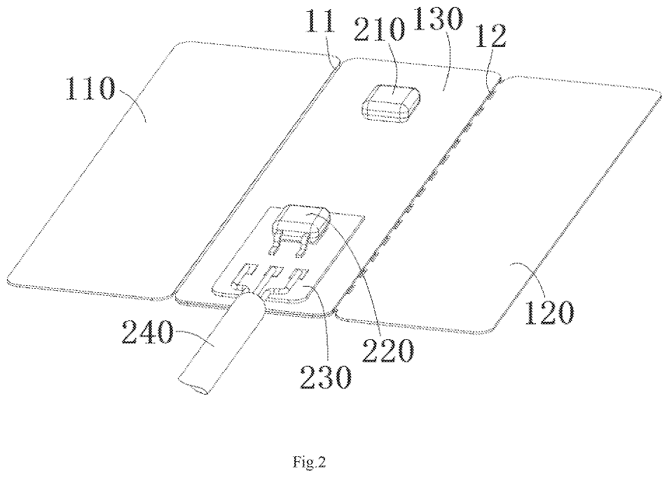

FIG. 1 shows a schematic diagram of a sealing tape according to an exemplary embodiment of the present invention. FIG. 2 shows a schematic diagram of adhering the electronic components 210, 220, 230 of the sensor to the middle area of the sealing tape shown in FIG. 1.

As shown in FIGS. 1 and 2, in the illustrated embodiment, the sensor mainly includes a sealing tape 100 and electronic components 210, 220 and 230. The sealing tape 100 can be folded along a first folding line 11 and a second folding line 12 formed on the sealing tape 100. The sealing tape 100 has a middle area 130 between the first folding line 11 and the second folding line 12, a first side area 110 at one side of the middle area 130, and a second side area 120 at the other side of the middle area 130.

As shown in FIG. 2, in the illustrated embodiment, the electronic components 210, 220 and 230 are adhered to the middle area 130 between the first folding line 11 and the second folding line 12 of the sealing tape 100.

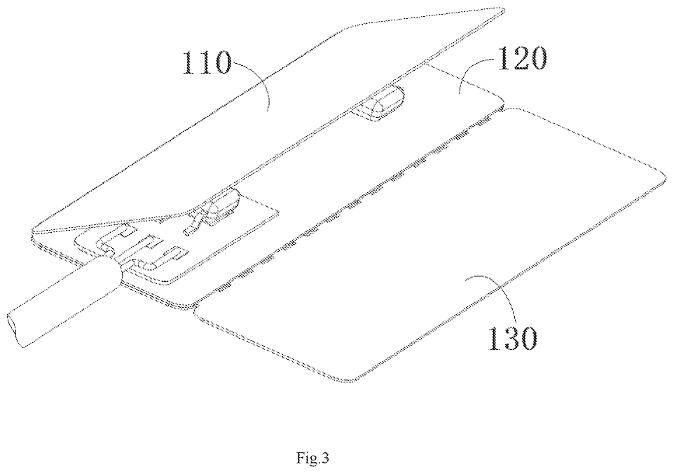

FIG. 3 shows a schematic diagram of folding and adhering the first side area 110 of the sealing tape 100 to the middle area 130 to which the electronic components 210, 220 and 230 have been adhered.

As shown in FIG. 3, in the illustrated embodiment, the first side area 110 of the sealing tape 100 is folded along the first folding line 11 and adhered to the middle area 130 to which the electronic components have been adhered. In this way, the electronic components 210, 220 and 230 are covered by the first side area 110. The periphery of the first side area 110 is pasted on the middle area 130, so that the electronic components 210, 220 and 230 are wrapped between the first side area 110 and the middle area 130.

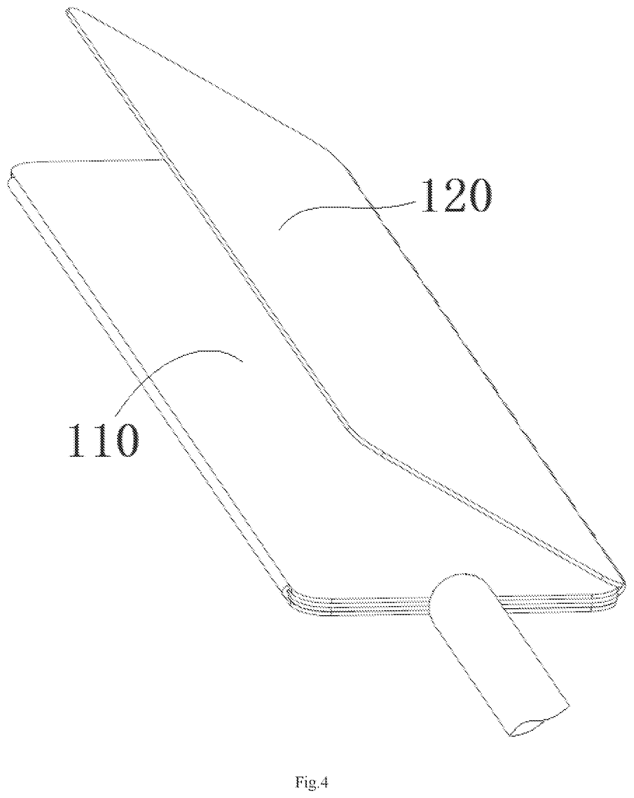

FIG. 4 shows a schematic diagram of folding and adhering the second side area 120 of the sealing tape 100 to the first side area 110 of the sealing tape 100.

As shown in FIG. 4, in the illustrated embodiment, the second side area 120 of the sealing tape 100 is folded along the second folding line 12 and adhered to the first side area 110 to hermetically wrap the electronic components 210, 220 and 230 in the sealing tape 100. The inner surface of the second side area 120 is adhered to the outer surface of the first side area 110. In this way, the second side area 120 and the first side area 110 are bonded together, so that the electronic components 210, 220 and 230 are hermetically wrapped in the sealing tape 100.

As shown in FIGS. 1 to 4, in the illustrated embodiment, the first folding line 11 and the second folding line 12 are preformed intermittent cut lines. However, the present invention is not limited to this. For example, in an exemplary embodiment of the present invention, the first folding line 11 and the second folding line 12 may be preformed creases or preformed indentations. In another exemplary embodiment of the present invention, the first folding line 11 and the second folding line 12 may be lines drawn on the surface of the sealing tape 100 in advance.

In an embodiment, the sealing tape 100 may include a strip-shaped waterproof substrate and an adhesive coated on the surface of the waterproof substrate. The waterproof substrate can be waterproof envelope paper, the adhesive is coated on one surface of the waterproof envelope paper.

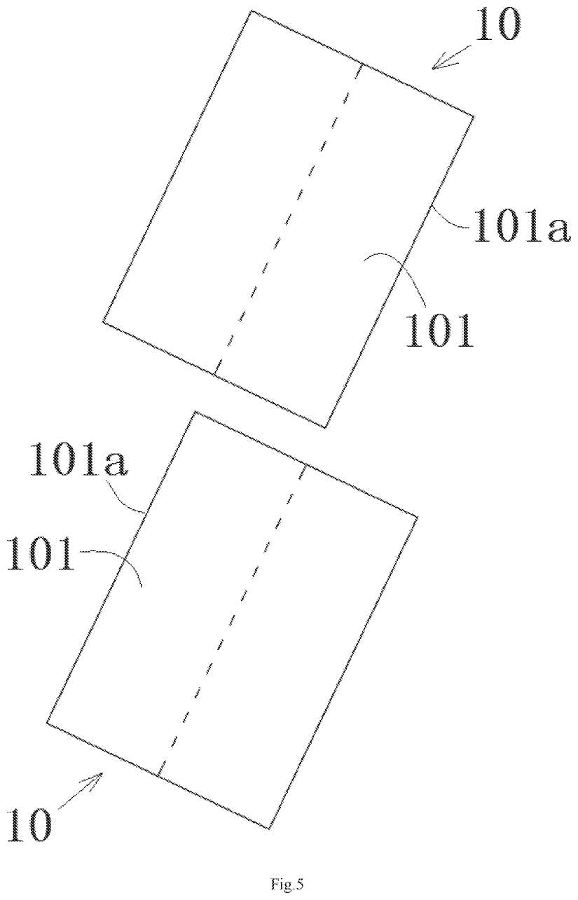

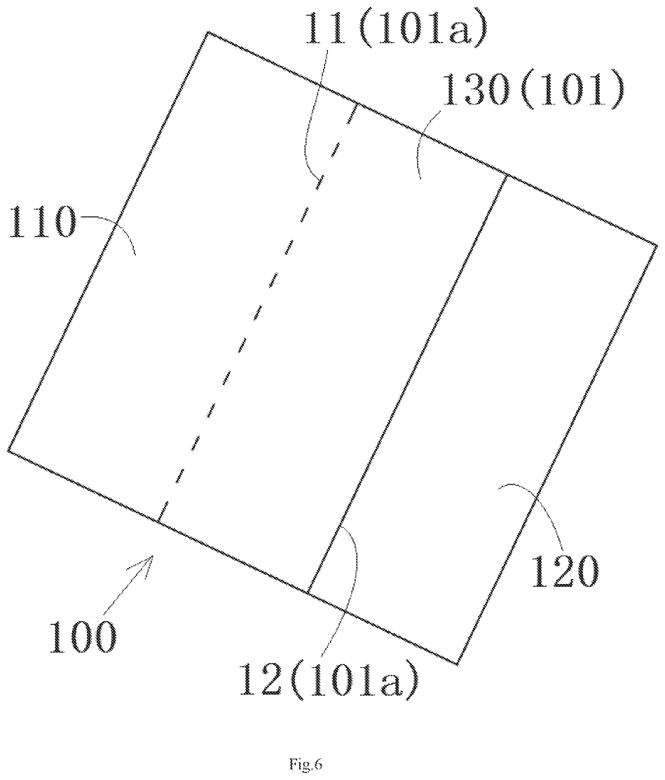

As shown in FIGS. 1 to 4, in the illustrated embodiment, the sealing tape 100 is a single piece of integral tape. In this way, it is very convenient to use. However, the present invention is not limited to the embodiments shown in FIGS. 1 to 4. For example, the sealing tape 100 may also be formed by bonding two pieces of sub adhesive tapes 10 (see FIGS. 5 and 6).

FIG. 5 shows a schematic diagram of two pieces of sub adhesive tapes 10 of a sealing tape 100 according to another exemplary embodiment of the present invention. FIG. 6 shows a schematic diagram of the sealing tape 100 bonded by two pieces of sub adhesive tapes 10 shown in FIG. 5.

As shown in FIGS. 5 and 6, in the illustrated embodiment, the sealing tape 100 is formed by bonding two pieces of sub adhesive tapes 10. Each piece of sub adhesive tape 10 includes a bonding area 101, and the bonding areas 101 of the two pieces of sub adhesive tapes 10 are overlapped and bonded with each other. The bonding area 101 of the sub adhesive tape 10 corresponds to the middle area 130 of the sealing tape 100.

As shown in FIGS. 5 and 6, in the illustrated embodiment, the first folding line 11 may be defined by the edge 101a of the bonding area 101 of one piece of sub adhesive tape 10, and the second folding line 12 may be defined by the edge 101a of the bonding area 101 of the other piece of sub adhesive tape 10. In this way, the manufacturing steps can be simplified without forming the first folding line 11 and the second folding line 12 in advance.

As shown in FIGS. 1 to 4, in the illustrated embodiment, the sensor may be a blood oxygen sensor for detecting blood oxygen saturation. However, the present invention is not limited to this, and the sensor may be any type of sensor that needs to seal the electronic component.

As shown in FIGS. 1 to 4, in the illustrated embodiment, the electronic components 210, 220, 230 include a light emitter 210 and a light receiver 220. The light emitter 210 is used to emit light, and the light receiver 220 is used to receive light emitted by the light emitter 210.

As shown in FIGS. 1 to 4, in the illustrated embodiment, the electronic components 210, 220 and 230 also include a circuit board 230. The light emitter 210 and the light receiver 220 are electrically connected to or mounted on the circuit board 230.

As shown in FIGS. 1 to 4, in the illustrated embodiment, the sensor also includes a cable 240. The end conductor of the cable 240 is electrically connected to the circuit board 230 and is hermetically wrapped in the sealing tape 100.

A method for manufacturing the illustrated sensor will be described in detail below with reference to FIGS. 1 to 4. The manufacturing method mainly includes the following steps:

S100: as shown in FIG. 1, providing a sealing tape 100 which has a first folding line 11 and a second folding line 12;

S200: as shown in FIG. 2, adhering the electronic components 210, 220 and 230 to the middle area 130 between the first folding line 11 and the second folding line 12 of the sealing tape 100;

S300: as shown in FIG. 3, folding a first side area 110 of the sealing tape 100 along the first folding line 11, and adhering the first side area 110 to the middle area 130 to cover the electronic components 210, 220 and 230 which have been adhered to the middle area 130; and

S400: as shown in FIG. 4, folding a second side area 120 of the sealing tape 100 along the second folding line 12, and adhering the second side area 120 to the first side area 110 to hermetically wrap the electronic components 210, 220 and 230 in the sealing tape 100.

As shown in FIG. 5, in an exemplary embodiment of the present invention, the aforementioned step S100 may include a step of:

S110: providing two pieces of sub adhesive tapes 10 each piece of which includes a bonding area 101. The bonding areas 101 of the two pieces of sub adhesive tape 10 are overlapped and bonded together to form a sealing tape 100.

In the above exemplary embodiments according to the present invention, the electronic components 210, 220 and 230 of the sensor are sealed and wrapped in the sealing tape 100 by folding the sealing tape 100, which can avoid the problem of side cracking of the sealing tape 100 and improve the waterproof sealing effect. Moreover, the present invention can realize the folding of sealing tape 100 through automatic equipment, which is suitable for mass production and reduces the cost.

It should be appreciated for those skilled in this art that the above embodiments are intended to be illustrative, and not restrictive. For example, many modifications may be made to the above embodiments by those skilled in this art, and various features described in different embodiments may be freely combined with each other without conflicting in configuration or principle.

Although several exemplary embodiments have been shown and described, it would be appreciated by those skilled in the art that various changes or modifications may be made in these embodiments without departing from the principles and spirit of the disclosure, the scope of which is defined in the claims and their equivalents.

As used herein, an element recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

What is claimed is:

1. A sensor, comprising:
a sealing tape having a first folding line and a second folding line; and
an electronic component adhered to a middle area between the first folding line and the second folding line, a first side area of the sealing tape is folded along the first folding line and adhered to the middle area to cover the electronic component, a second side area of the sealing tape is folded along the second folding line and adhered to the first side area to hermetically wrap the electronic component in the sealing tape.

2. The sensor of claim 1, wherein the first folding line and the second folding line are preformed intermittent cut lines, creases, or indentations.

3. The sensor of claim 1, wherein the sealing tape has a waterproof substrate that is strip-shaped and an adhesive coated on a surface of the waterproof substrate.

4. The sensor of claim 3, wherein the waterproof substrate is a waterproof envelope paper.

5. The sensor of claim 4, wherein the adhesive is coated on one surface of the waterproof envelope paper.

6. The sensor of claim 1, wherein the sealing tape is a single piece of integral adhesive tape.

7. The sensor of claim 1, wherein the sealing tape is formed by bonding a pair of pieces of sub adhesive tape.

8. The sensor of claim 7, wherein each piece of the sub adhesive tape includes a bonding area, and the bonding areas of the pieces of sub adhesive tape are overlapped and bonded with each other.

9. The sensor of claim 8, wherein the bonding areas of the sub adhesive tape corresponds to the middle area of the sealing tape.

10. The sensor of claim 9, wherein the first folding line is defined by an edge of the bonding area of one piece of sub adhesive tape, and the second folding line is defined by an edge of the bonding area of the other piece of sub adhesive tape.

11. The sensor of claim 1, wherein the sensor is a blood oxygen sensor for detecting blood oxygen saturation.

12. The sensor of claim 11, wherein the electronic component includes a light emitter for emitting light and a light receiver for receiving the light emitted by the light emitter.

13. The sensor of claim 12, wherein the electronic component includes a circuit board.

14. The sensor of claim 13, wherein the light emitter and the light receiver are electrically connected to or mounted on the circuit board.

15. The sensor of claim 14, further comprising a cable, an end conductor of the cable is electrically connected to the circuit board and sealed and wrapped in the sealing tape.

16. A method of manufacturing a sensor, comprising:
providing a sealing tape which has a first folding line and a second folding line;
adhering an electronic component to a middle area between the first folding line and the second folding line of the sealing tape;
folding a first side area of the sealing tape along the first folding line and adhering the first side area to the middle area to cover the electronic component; and
folding a second side area of the sealing tape along the second folding line and adhering the second side area to the first side area to hermetically wrap the electronic component in the sealing tape.

17. The method of claim 16, wherein the providing step includes providing a pair of pieces of sub adhesive tape each having a bonding area, the bonding areas of the pieces of sub adhesive tape are overlapped and bonded with each other.

18. The method of claim 17, wherein the first folding line is defined by an edge of the bonding area of one piece of sub adhesive tape, and the second folding line is defined by an edge of the bonding area of the other piece of sub adhesive tape.
